(19) United States
(12) Patent Application Publication
(10) Pub. No.: US 2010/0198051 A1
(43) Pub. Date: Aug. 5, 2010

(54) ACTUATORS

(75) Inventors: Michael Ulrich Lamperth, Surrey (GB); Ian Robert Young, London (GB); Haytham Elhawary, London (GB); Tsz Ho Tse, London (GB)

Correspondence Address: FRASER CLEMENS MARTIN & MILLER LLC, 28366 KENSINGTON LANE, PERRYSBURG, OH, 43551, US

(73) Assignee: IMPERIAL INNOVATIONS LIMITED, London (GB)

(21) Appl. No.: 12/515,207

(22) PCT Filed: Nov. 15, 2007
(86) PCT No.: PCT/GB2007/004362

§ 371 Date: Mar. 31, 2010

(30) Foreign Application Priority Data
Nov. 15, 2006 (GB) 0622725.0

(51) Int. Cl. A61B 19/00; F01D 15/06; A61B 5/05; G06F 19/00; B25J 18/00

(52) U.S. Cl. 600/415; 415/122.1; 600/410; 700/245; 74/490.01

(57) ABSTRACT

An actuator comprises an input shaft (42), a turbine system (38) coupled to the input shaft, air inlet means (32, 34) arranged to direct air flowing through it towards the turbine system to rotate the input shaft, an output shaft (50), and a gearing system (48) connecting the input shaft (42) to the output shaft (50) so that the turbine system (38) can drive the output shaft (50) via the gearing system (48). The air inlet means defines two different flow paths for air whereby the output shaft (50) can be driven in both directions.

The present invention relates to actuators, and in particular to actuators arranged to be powered by pressurised air or other gas. The actuators of the present invention have particular application in the field of robots, for example for use within MRI scanners, but can also be used in a wide variety of other applications.

The good soft tissue contrast of MRI makes it a very sensitive and powerful diagnostic technique. The extensive advantages and capabilities offered by this imaging modality are well known as are the restrictions imposed on the MRI environment by the magnetic fields which are required to obtain an image. Due to these interactions, MR safety and compatibility must be verified before any device can be introduced into the MR scanner room.

There are various approaches to manipulating devices, such as probes, within an MRI scanner. One approach is to locate a surgical robot within the scanner, but to control it using actuators located outside the scanner. This helps to prevent the actuators, typically electric motors, from interfering with the scanner image. However another approach is to locate the robot actuators with the robot within the scanner. In this case the actuator needs to meet a number of criteria. It must be small, as the opening of a typical MRI scanner is about 600 mm and the robot must fit inside this with the patient. It should not cause potential hazard to the patient, so high voltage (HV) electrical power supplies and hydraulic fluids are not desirable. High pressure air is also undesirable, and for safety considerations air pressures in practice are often limited to less than 2 bar to avoid potential for an air embolism. The actuator needs to be MR compatible, making electric motors and a large number of standard engineering materials unsuitable. Rotary actuators generally need to produce torque in both directions. The output must be at a useful speed, and the device must be controllable with a high degree of accuracy.

The present invention provides an actuator comprising an input shaft, a turbine system coupled to the input shaft, gas inlet means arranged to direct gas flowing through it towards the turbine system to rotate the input shaft, an output shaft, and a gearing system connecting the input shaft to the output shaft so that the turbine system can drive the output shaft via the gearing system, wherein the gas inlet means defines two different flow paths for gas whereby the output shaft can be driven in both directions.

The input shaft may be of non-magnetic material, such as aluminium or glass. The turbine may also be of non-magnetic material.

The present invention further provides an actuation system comprising an actuator according to the invention, and a control system arranged to control the supply of air to the air inlet means thereby to control the actuator.

The control system is preferably arranged to limit the gas pressure at at least one point in the system to a maximum value, which may be 5 bar, or even 2 bar.

The present invention still further provides a robot system comprising a control element, an actuation system according to the invention arranged to receive a demand signal and control the position of the control element, and an operator input arranged to be controlled by a user to control the demand signal, whereby the user can control the position of the control element.

The present invention still further provides an MRI scanning system including a scanner volume and magnets arranged to vary the magnetic field in the scanner volume, and a robot system according to the invention wherein the actuator and the control element are located within the scanner volume.

As well as applications in MRI scanning systems and other medical applications, the actuator of the invention has utility in other (non-MR) application areas where EM motors are undesirable or represent a safety risk. For example, in environments where explosion or fire hazards such as powder suspensions or flammable vapours are present.

Preferred embodiments of the present invention will now be described by way of example only with reference to the accompanying drawings in which:

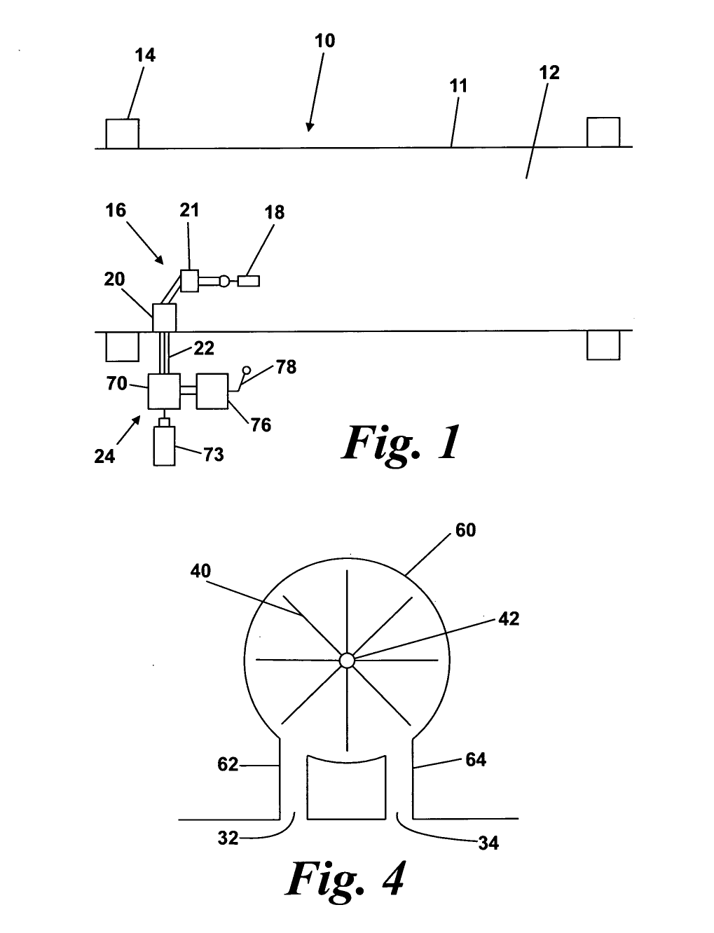

FIG. 1 is a schematic view of an MRI scanner with a robot located within it;

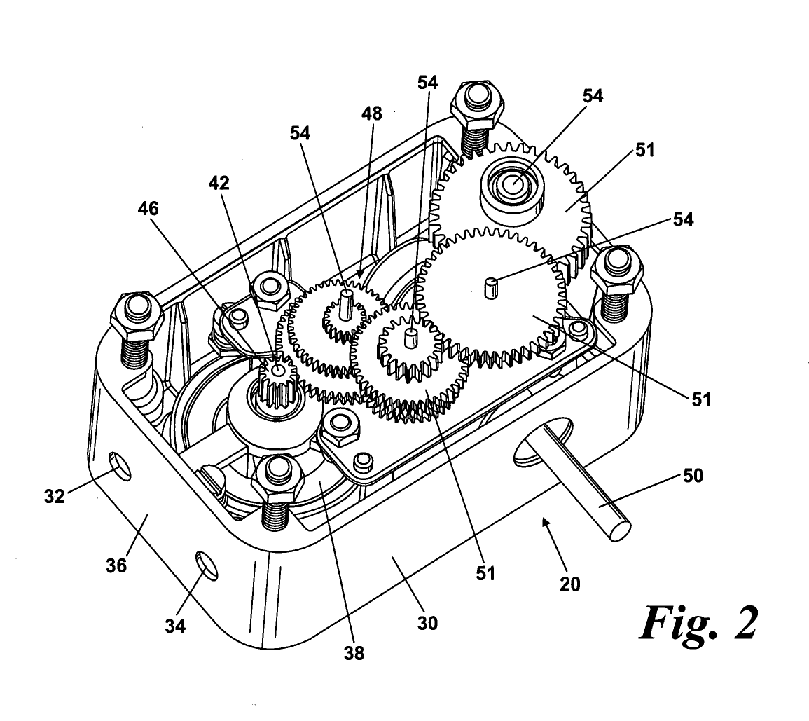

FIG. 2 is perspective view of an actuator of the robot of FIG. 1;

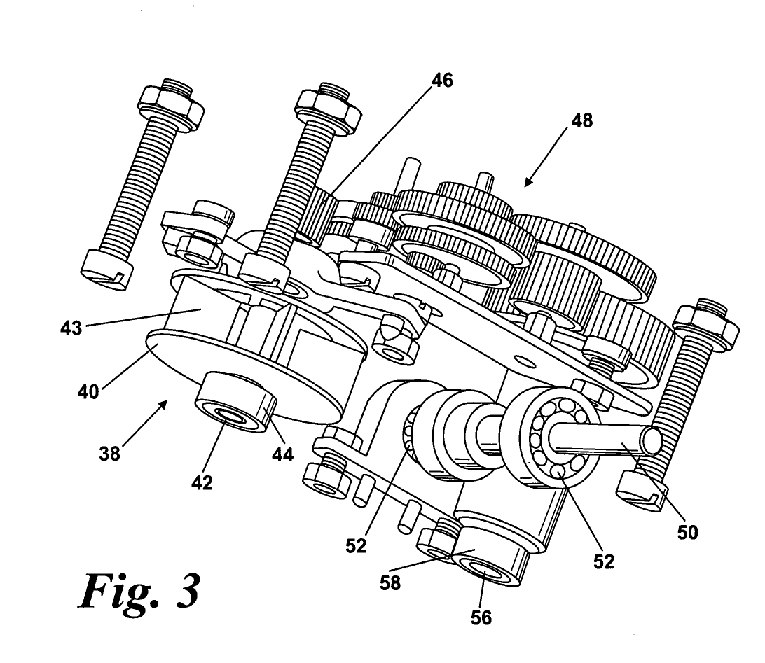

FIG. 3 is a perspective view of the internal components of the actuator of FIG. 2;

FIG. 4 is a schematic plan view of the turbine rotor and inlet nozzles of the actuator of FIG. 2;

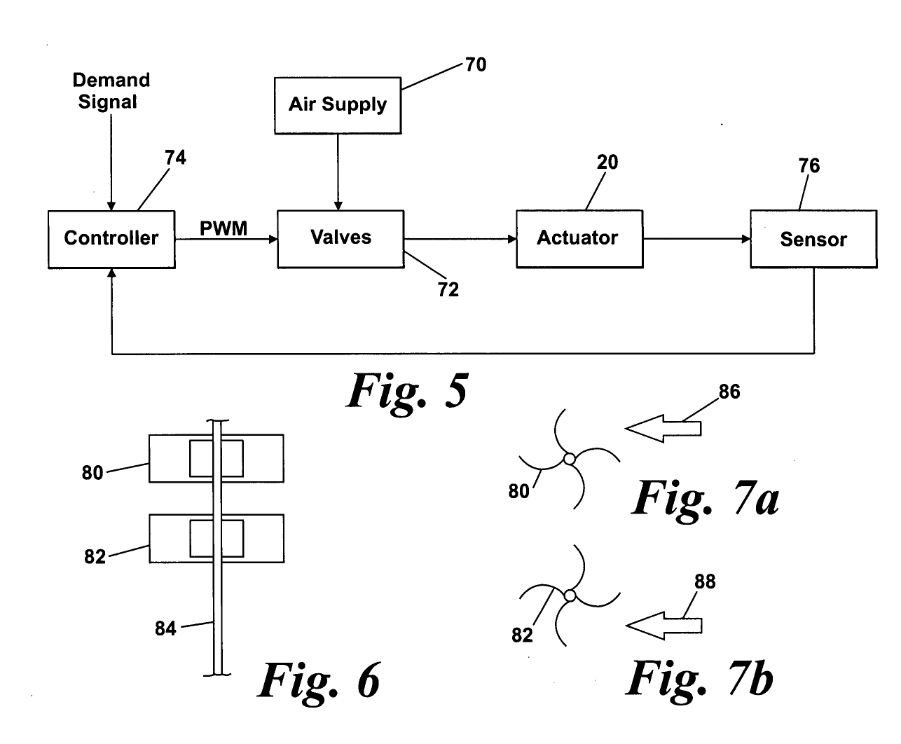

FIG. 5 is a diagram of the actuator of the robot of FIG. 1 and its control system;

FIG. 6 is a vertical section through a turbine system of an actuator according to a second embodiment of the invention;

FIGS. 7a, and 7b are horizontal sections through the turbine system of FIG. 6;

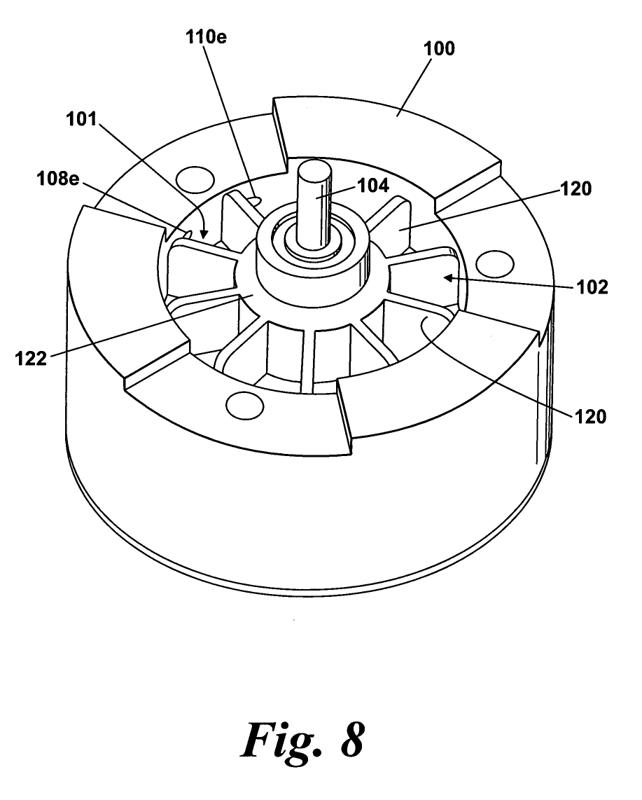

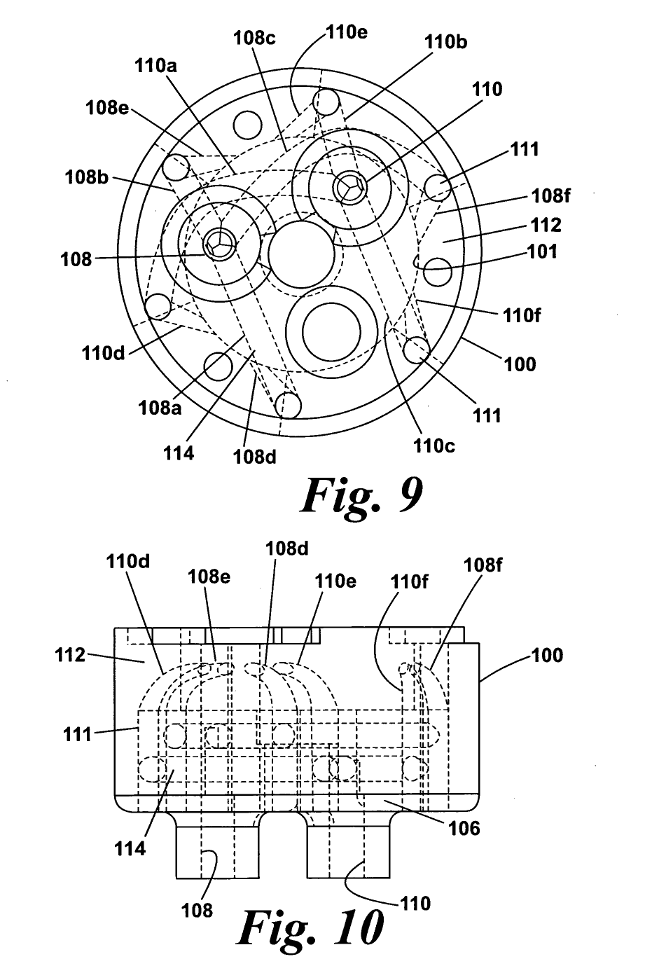

FIG. 8 is a perspective view of a turbine system according to a further embodiment of the invention;

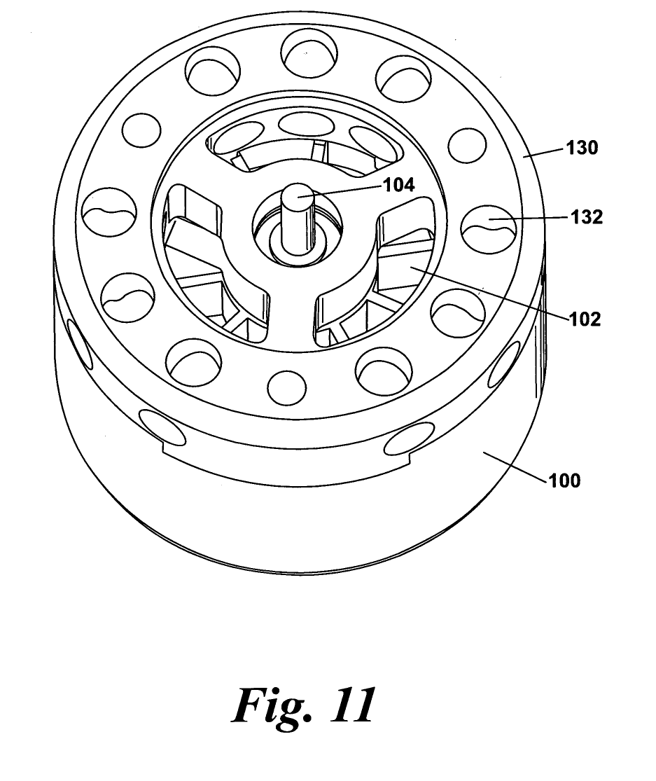

FIG. 9 is a transverse section through part of the turbine system of FIG. 8;

FIG. 10 is a side view of part of the turbine system of FIG. 8;

FIG. 11 is a perspective view of the turbine system of FIG. 8 with a porting section attached;

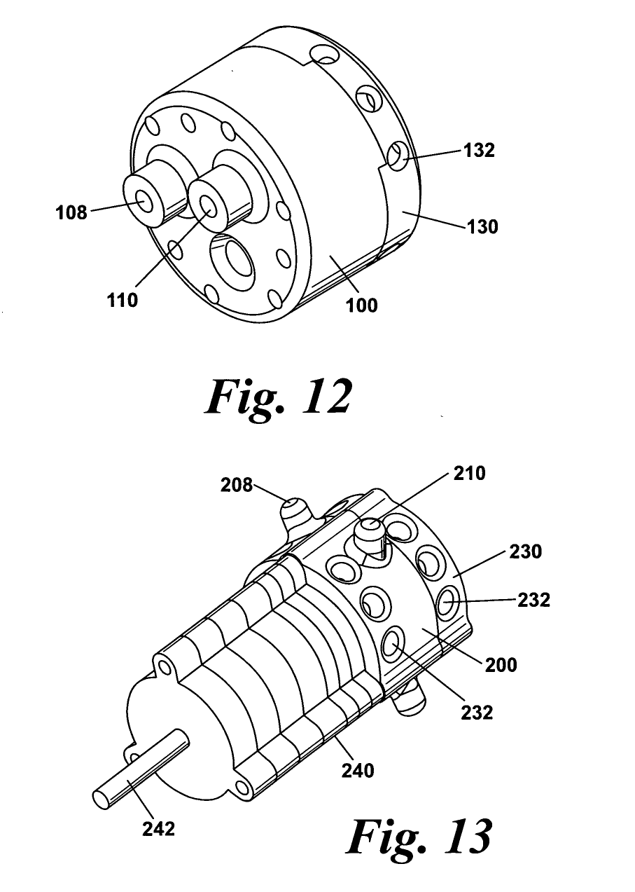

FIG. 12 is a perspective view of the turbine system of FIG. 9;

FIG. 13 is a perspective view of the turbine system according to a further embodiment of the invention with a gearbox connected to it;

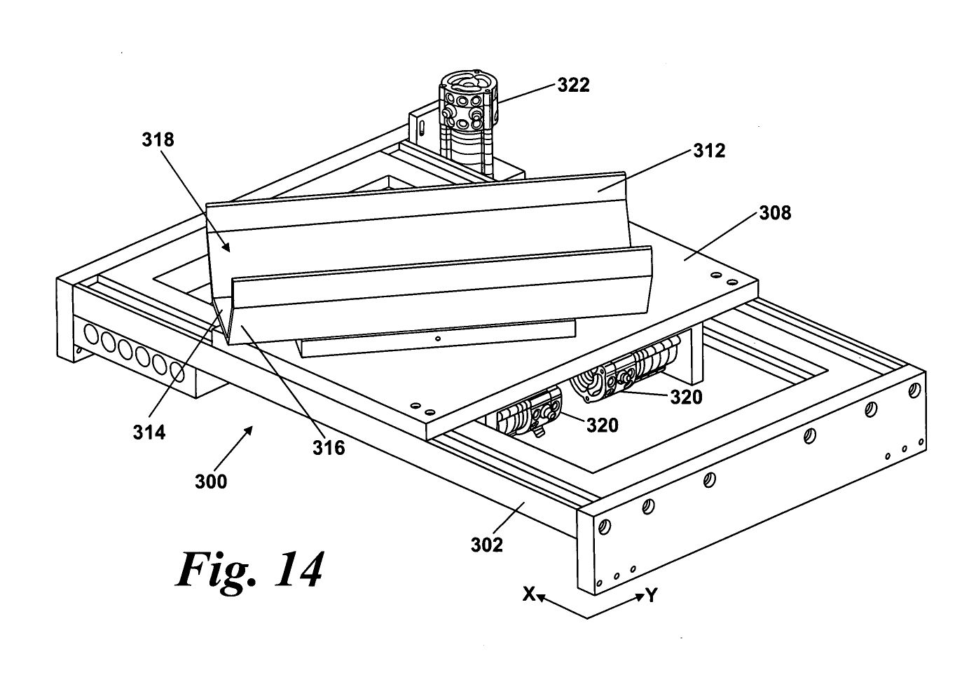

FIG. 14 is a perspective view of a manipulator according to a further embodiment of the invention;

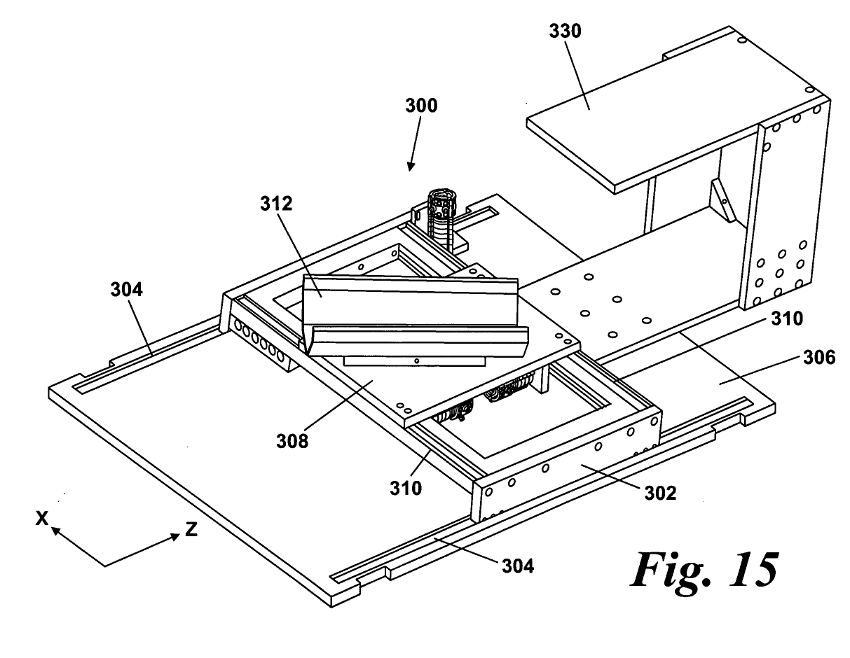

FIG. 15 is a perspective view of the manipulator of FIG. 14 and associated support;

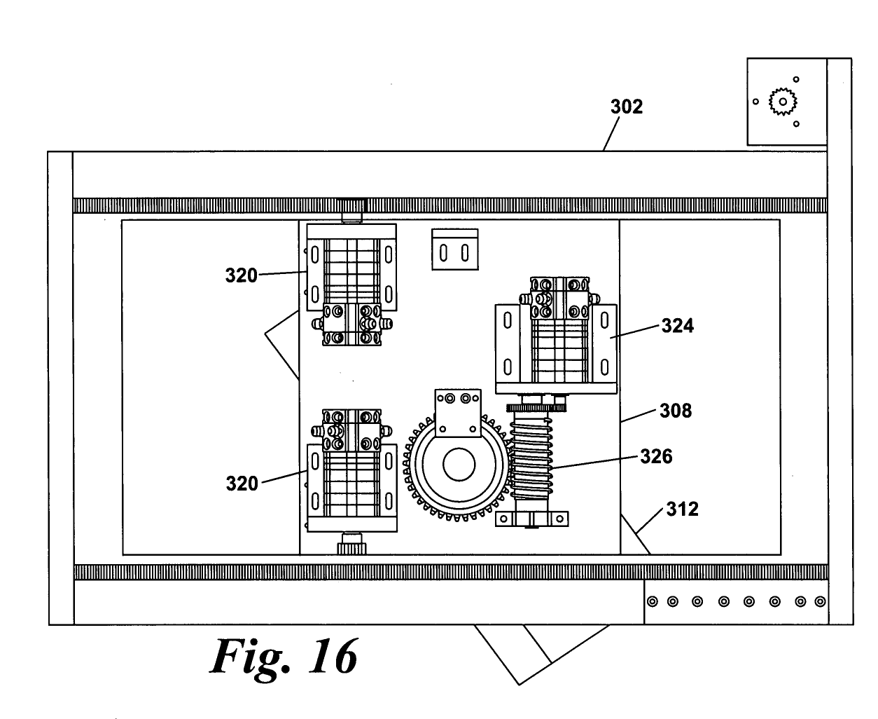

FIG. 16 is a view of the underside of the manipulator of FIG. 14;

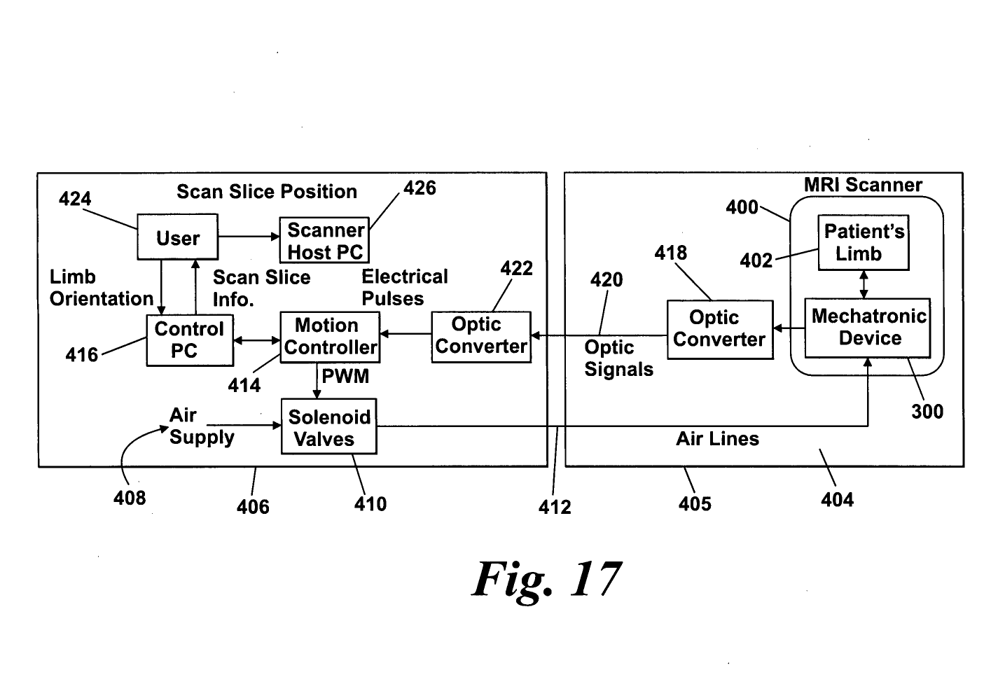

FIG. 17 is a diagram of the a scanning system including the manipulator of FIG. 14;

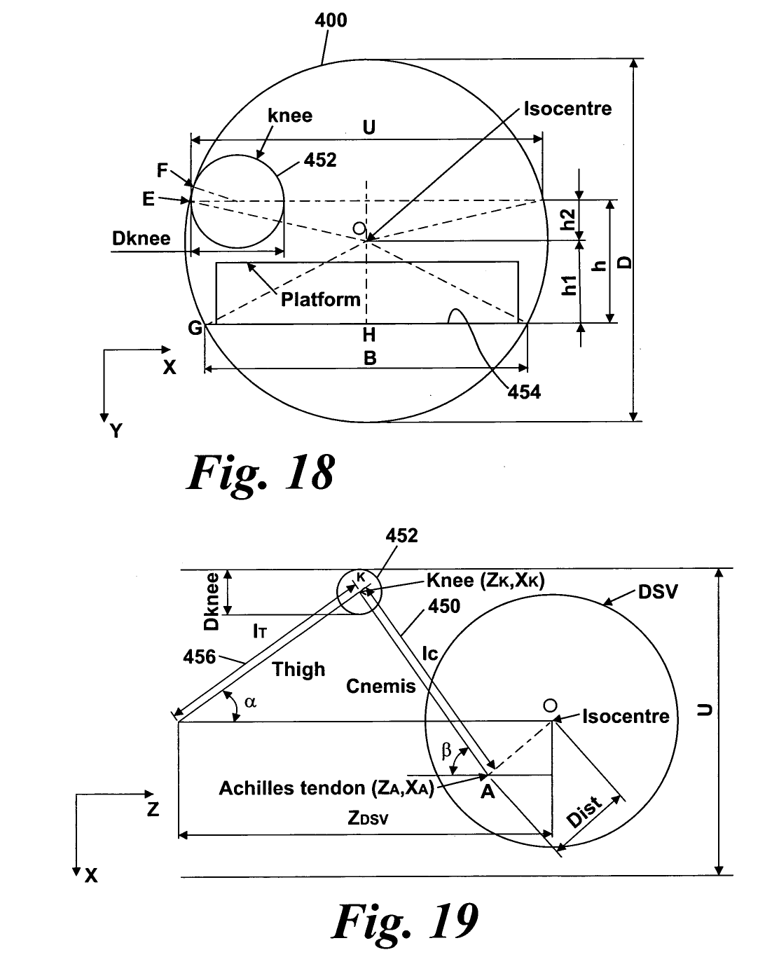

FIG. 18 is a schematic transverse section through the scanner bore of the scanning system of FIG. 17; and

FIG. 19 is a schematic longitudinal section through the scanner bore of the scanning system of FIG. 17.

Referring to FIG. 1, an MRI scanner 10 comprises a housing 11 defining a scanner volume in the form of a scanner bore 12 and magnets 14 arranged to vary the magnetic field within the scanner bore 12 in known manner. A surgical robot 16 is located in the scanner bore 12 and is arranged to support a surgical device, in this case probe 18. The robot 16 includes a number of actuators 20, 21 each of which is controlled by pressurised gas supplied along a respective pair of supply tubes 22 from an actuator control system 24, which is preferably located outside the scanner room. If the control system is located inside the room it will need to be shielded to prevent interference with the MR scanner. The actuators 20, 21 are each arranged to control one degree of freedom of the robot 16 so that together they can control the position of the probe 18.

Referring to FIGS. 2 and 3, one of the actuators 20 will now be described. The actuator or motor 20 comprises a housing 30 having two gas inlet ports 32, 34 in its end wall 36. A turbine system 38 comprises a turbine rotor 40 mounted on an input shaft 42, and having a number of rotor blades 43. The input shaft is supported by bearings 44 so that it can rotate, and is rigidly connected to the turbine rotor 40 so as to rotate with it. A gear wheel 46 is mounted on the opposite end of the input shaft to the rotor 40. The gear wheel 46 forms the input of a gear box 48. An output shaft 50 is rotatably mounted on bearings 52 and is driven by the gearbox 48. The gearbox comprises four support shafts 54 mounted in the housing, all parallel to the input shaft 42, and a number of gear wheels 51 supported on the shafts 54. One of the support shafts 54 forms a drive a gearbox output shaft 56 which is supported in bearings 58 and which in turn drives the actuator output shaft 50 by means of a worm gear arrangement. The gear on shaft 56 is a worm gear that engages with a gear on shaft 50. The gearbox in this embodiment has a gearing ratio of 1:4616.

Referring to FIG. 4, the turbine rotor 40 is surrounded by wall 60. A nozzle 62, 64 extends from each of the inlet ports 32, 34 to a respective aperture through the wall 60. The nozzles 62, 64 are approximately parallel and located on opposite sides of the axis of rotation of the rotor 40 so that each of them is arranged to direct the flow of gas tangentially onto the rotor. The rotor blades 43 are flat on both sides and extend radially outwards from the rotor axis, and the two nozzles 62, 64 direct gas at opposite sides of the blades 43 so that they provide driving torques to the rotor 40 which tend to drive the rotor 40 in opposite directions respectively. Additional nozzles may be added to increase the torque on the rotor. For example two or three nozzles could be provided for producing torque in each direction.

Referring to FIG. 5, a control system for the actuators 20, 21 is comprised of a pressurised air supply 70, two valves 72 for each actuator, each valve being arranged to control the supply of air to a respective one of the nozzles 62, 64 proximal to the turbine rotor 40, a controller 74 with software to control the aperture of the valves via pulsed width modulation (PWM) and sensing equipment 76 to detect the position of the output of each of the actuators 20, 21. A demand signal, which may be generated by a user input, or by control algorithm, is input to the controller 74 and introduced in the software of the controller for each degree of freedom of the robot. This demand signal is compared to the signal produced by the sensing device, which is fed into the controller, and as a result of the divergence of these two values, a PWM output is sent to set the aperture of the valves. The duty ratio of the PWM signal is predetermined by a standard Proportional Integral Derivative (PID) algorithm. This control system enables the control of both valves for each actuator independently so that it can regulate the air supply to each of the nozzles of each of the actuators and thus control the direction and magnitude of the output torque of each of the actuators.

When a sudden braking force is required to stop the actuator the open nozzle can be closed and the other nozzle opened so as to brake the turbine. If a high holding torque is needed, both valves can be fully opened together.

In this embodiment the air is supplied from the air supply at a controlled pressure of 1.5 bar and the turbine speed is limited to 30,000 rpm. This generates an output torque of up to 0.5 Nm and an unloaded speed of 6 revolutions per minute.

If more than one nozzle is provided for each direction, then each nozzle may have its own control valve so that the torque can be controlled by opening or closing each of the valves, and hence each of the nozzles, independently.

Referring to FIG. 6, in a further embodiment of the invention the turbine system comprises a double impulse turbine having two rotors 80, 82 mounted on the same input shaft 84. In this case the turbine rotors 80, 82 are both asymmetrically shaped, having curved blades 84. The blades on the two rotors 80, 82 are curved in opposite directions so that, viewed axially as in FIGS. 7a and 7b, the concave sides of the blades of one rotor 80 face in the clockwise direction and the concave sides of the blades of the other rotor 82 face in the anticlockwise direction. For each rotor the respective nozzle 86, 88 is arranged to direct air at the concave side of the blades, so that the rotors are arranged to drive the input shaft in opposite directions.

In a modification to the embodiment of FIG. 6, the two rotors are not mounted on a common shaft, but are mounted on separate support shafts each of which is coupled to a common input shaft of the gearbox, for example by suitable gearing.

It will be appreciated that the gearbox of the embodiments described above can be substituted with a range of different types of gearbox, depending on the application for which the actuator is intended. In one modification to the embodiment of FIGS. 2 and 3, the turbine rotor is attached to a modular (multi-stage) epicyclic gear system, for example as supplied by Tamiya under part number 72001. In this way increased gear ratios can be obtained by adding more modules. This reduces noise and backlash and can give a very high gear ratio in a compact space.

Referring to FIG. 8, a turbine system according to a further embodiment of the invention comprises a turbine housing 100, and a turbine rotor 102 supported within a cavity 101 within the housing on an input shaft 104. Referring also to FIGS. 9 and 10, the housing 100 is cylindrical having an end wall 106 with two inlet ports 108, 110 formed in it, and a cylindrical wall 112. The housing 100 has inlet ducts 114 formed in it which lead from the inlet ports to the inside of the cylindrical wall where they direct gas onto the turbine rotor 102. As can best be seen in FIG. 9, each of the inlet ports 108, 110 leads to a group of three branch ducts 108a, 108b, 108c, 110a, 110b, 110c formed in the end wall 106 of the housing. Each group of branch ducts leads to three respective axial ducts 111 formed in the cylindrical wall, which in turn lead to three nozzles or outlets 108d, 108e, 108f, 110d, 110e, 110f which are arranged to direct gas inwards onto the turbine rotor 102. Each of these groups of nozzles is evenly spaced around the cylindrical housing at 120° intervals. The nozzles of one of the groups 108d, 108e, 108f all point at an angle which is tangential to the turbine axis, and in the same direction (clockwise as seen in FIG. 9) about the axis, and the nozzles of the other group 110d, 110e, 110f are similarly spaced, and angled, but point tangentially in the opposite direction (anti-clockwise as seen in FIG. 9) about the turbine axis.

Referring back to FIG. 8, the turbine rotor 102 has a number of flat blades 120 each of which extends radially outwards from a turbine hub 122 which in turn is rigidly connected to the input shaft 104. If gas is introduced into one of the inlet ports 108, it will be distributed to the three nozzles 108d, 108e, 108f connected to that inlet port. The nozzles will direct the gas against the turbine blades 102 to drive the turbine in one direction. If gas is introduced into the other inlet port 110, then it will be distributed to the other group of nozzles 110d, 110e, 110f and the turbine 102 will be driven in the opposite direction.

Gas leaving the turbine cavity 101 flows away from the turbine 102 axially, in the direction away from the end wall 106. Referring to FIGS. 11 and 12, an outlet porting section 130 is mounted on the open end of the housing 100 and has a number of outlet ducts 132 through it opening into the turbine cavity at a position axially offset from the turbine blades and connecting the turbine cavity 101 to a series of outlet ports in its radially outer wall. This allows the exhaust gas from the turbine 102 to escape when other modules, for example gearing modules, are attached to the open end of the housing 100.

In use, gas is introduced through the converting nozzles 108d, 108e, 108f, 110d, 110e, 110f which are tapered to speed up its velocity before hitting the turbine rotor 102. The internal and kinetic energy of 0.1 MPa air are converted to rotational energy at the shaft, after which a gear train (ratio 2000:1) is adapted to provide a high torque up to 0.3 Nm and maximum speed of 24 rpm. The gear train comprises plastic gears, bearings with glass balls and an aluminium shaft. Keeping the operating pressure at around 1 bar ensures the safety even if air leakage occurs near the patient.

Referring to FIG. 13, in a further embodiment the turbine housing 200 is provided with two inlet ports 208, 210 for each direction, and outlet ports 232 in the turbine housing 200 as well as the porting section 230. It is connected to a modular gearbox 240 which has an output shaft 242.

Referring to FIGS. 14 and 15, a manipulator 300 for controlling movement of a patient's leg within an MR scanner comprises a lower carrier 302 supported on a pair of rails 304 on a base 306. An upper carrier 308 is supported on a further pair of rails 310 on the lower carrier. The two sets of rails 304 310 are perpendicular to each other so the lower carrier 302 can move in one direction, referred to herein as the Z direction, on the base 306, and the upper carrier 308 can move in a perpendicular direction, referred to herein as the X direction, on the lower carrier 302. A leg support 312 is mounted on the upper carrier 308 on a rotatable support so that it can rotate about a vertical axis relative to the upper carrier 308. The leg support 312 includes a base 314 and side walls 316 which define a horizontal channel 318 in which the cnemis of a patient can be supported.

Referring also to FIG. 16, an air motor 322 is arranged to drive the lower carrier 302 in the Z direction along the base 306, a further pair of air motors 320 is arranged to drive the upper carrier 308 along the lower carrier 302, and a further air motor 324 is arranged, via a worm drive 326, to rotate the leg support 312 about its vertical axis of rotation. It will therefore be appreciated that the leg support 312 can be moved anywhere, and in any direction, in the horizontal plane, within the limits of the carriers 302, 308, and rotated about its vertical axis to any orientation.

Referring back to FIG. 15, the manipulator system further comprises an upper leg support 330 which is above the leg support 312, and arranged to support a second leg of a patient, while the first leg of the patient is supported in the leg support 312. This is so that a patient can lie on their side, with the cnemis of their lower leg supported in the rotatable leg support 312, and their other, upper leg supported on the upper leg support 330 so that it is clear of the lower leg, and the lower leg is free to be moved by the manipulator.

Referring to FIG. 17, the manipulator 300, which in general terms can be referred to as a mechatronic device, is located within the scanner bore 400 of an MRI scanner, so that it can support the patient's limb 402, in this case a leg. The scanner is located in a scanner room 404 which is surrounded by shielding 405 to shield it from magnetic interference. A control room 406 is located outside, and near to, the scanner room 404 and the main control elements of the system are located in the control room 406. Specifically an air supply 408 is provided in the control room, as is a valve block 410 containing solenoid valves for controlling the supply of air to the air motors 320, 322, 234 of the manipulator 300. Air lines 412 connect the valve block 410 to the manipulator 300. A motion controller 414 provides PWM signals to the valve block 410 to control the valves, itself being controlled by a control PC 416. The manipulator includes position sensors which generate electrical signals indicative of the position of the leg support 132 in the Z and X directions and its orientation. These signals are converted to optical signals in an optic converter 418 in the scanner room 404, and transmitted over optical cables 420 to a further optical converter 422 in the control room 406 which converts them back into electrical signals for input to the motion controller 414. This provides a closed loop control of the position and orientation of the leg support 312 by the motion controller 414. A user input 424 enables a user to input a required limb orientation to the control PC 416, and also communicates with a scanner host PC 426 which can control the orientation of the image slices recorded by the scanner. This allows coordination between the orientation and position of the limb to be imaged, and the orientation of the imaging slices which allows the quality of the images to be maximised using the magic angle as will be described in more detail below. The user input may, instead of having a required limb position or orientation, input other requirements such as the part of the limb which is to be imaged. In any case the control PC 416 is arranged to determine an end position and a trajectory for achieving that end position from the information which is input by the user. It is then arranged to communicate this to the motion controller 414 which controls the valves in the valve block to operate the motors to control movement of the manipulator.

The aim of the control of the patient's limb is to achieve optimal orientation of the object to be imaged, in this example the Achilles tendon, relative to the main field of the MRI scanner. It is know that optimal imaging is achieved when the target object is orientated at 55° to the main magnetic field. However this has to be achieved within the constraints on movement of all parts of the patient's leg, including the thigh and cnemis, imposed by the scanner bore, which is typically of the order of 60 cm in diameter.

Referring to FIGS. 18 and 19, when the patient is in the scanner bore 400, lying on one side with the cnemis 450 of their lower leg supported in the support 312, the knee 452 of their lower leg, of which the Achilles tendon is to be imaged, will be close to the side of the scanner bore 400. The patient's hip position will be fixed relative to a table 454, and the patient's thigh 456 will therefore pivot about that position.

The aim of the manipulator system is to position a target tendon of a patient at the magic angle (about) 55° relative to the main field of an MRI scanner, which is in the Z direction, by controlling the movement of the patient's thigh 456 and cnemis 450. Some features of the positioning algorithm and the manipulator design can be descried as follows.

1. The system is arranged to be used inside the bore of a conventional cylindrical MR scanner with strength of 1.5 or 3 Tesla independent of the model and make.

2. Due to the spatial constraints of the scanner, the system is designed to be small, compact and cooperated well with the patient's limbs in an MR scanner bore of 60 cm. The height of the system is limited as a tall system restricts the movement area of a leg, causing knee collision to the inner wall of the scanner at a premature angle.

3. The system is able to offer users the best trajectory for the limb motion with the tissue of interest nearest to the centre of the Diameter Spherical Volume (DSV), which is the volume of the best field homogeneity.

4. The system can provide 2 dimensional movements along the XZ plane of a scanner at any point in the DSV. For rotational motion, the system is capable of rotating a target at least 60° in clockwise and anticlockwise direction relative to the main field (Z direction).

5. Components used are all non-ferromagnetic to ensure minimal sizes of artifacts generated. The Signal to Noise Ratio (SNR) reduction caused by electronics has been quantified to guarantee that the system does not affect the image quality during operation.

The algorithm for controlling movement uses various parameters which are defined as follows:

### Parameters Used in Actual View in FIG. 18

h Vertical distance from the centre of the knee joint to the scanner table
h₁ Vertical distance from the scanner table to the isocentre
h₂ Vertical distance from the centre of the knee joint to the isocentre
D Diameter of the scanner
B Width of the scanner table
U Maximum horizontal distance available for the cnemis to be oriented nearest to the actual plane
D_{Knee} Diameter of the leg at the knee

### Parameters Used in Sagittal View in FIG. 19

l_{T} Length of the thigh
l_{C} Length of the cnemis
α Angle of the femur relative to the main field of a scanner
β Angle of the tibia relative to the main field of a scanner
z_{DSV} Distance from the centre of the hip joint to the isocentre
[z_{K} x_{K}] z, x positions of the centre of the knee joint relative to the centre of the hip joint
[z_{A} x_{A}] z, x positions of the centre of the Achilles tendon relative to the centre of the hip joint

Placing the Achilles tendon at the magic angle requires the robotic system moving the thigh and cnemis together as shown in FIG. 19 and following a trajectory to minimise the distance from the tendon to the centre of DSV. With the ability to position a patient's leg anywhere in the DSV, the inverse kinematics of the 3 DOF manipulator system is arranged to control the movements of the thigh 456 and cnemis 450 by controlling the movement of the lower part of the cnemis or ankle. In order to do this the control system is arranged to define one or more relationships between movements of the cnemis and thigh which define how the thigh will move in response to movements of the cnemis, and also define movements which are acceptable and movements which are unacceptable (or impossible) taking into account the anatomy of the leg and the need to avoid straining the thigh and knee joints. This provides conditions on the movements of the leg support which are taken into account when selecting a trajectory towards the desired end position. The positioning algorithms are arranged to:

1. Decide the necessary movements of the leg support in X and Z directions to position the tendon at the right place with desired orientation and minimised distance from the isocentre O.

2. Find out the kinematic relationship between X, Z, and rotational axis of the system to have the tendon, thigh, and the cnemis follow a trajectory to achieve their optimal positions. Understanding the kinematic relationships assists the implementation of a control scheme on the air motors.

3. Calculate the minimum distance of the tendon from the isocentre at a particular orientation of the tendon.

4. Estimate the necessary movement of the scanner table to further reduce the minimum distance if a better image quality is needed.

5. Decide the [z_{A} x_{A}] coordinate of the tendon and the rotational angle of the tendon relative to the main field, which is used for image slice tracking and orientation of the tendon during scanning.

The algorithm used in this embodiment includes the following steps.

1). The position [z_{A} x_{A}] of the Achilles tendon at desired orientation β is calculated using:

[z_{A} x_{A}]=[(l_{T} cos α_{min dist}+l_{C} cos β) (l_{C} sin β−l_{T} sin α_{min dist})]

2). The minimum distance from the Achilles tendon to the isocentre is given by:

$$Dist = \sqrt{\begin{matrix}{( {l}_{C} sin β - {l}_{T} sin {α}_{min dist} )}^{2} + \\ {[ {z}_{DSV} - ( {l}_{T} cos {α}_{min dist} + {l}_{C} cos β ) ]}^{2} + {h}_{2}^{2}\end{matrix}}$$

3). The minimum distance can be further reduced if moving the table 454 in the Z direction is allowed by

$$Δ {z}_{table} = {z}_{DSV} - ( {l}_{T} cos {α}_{min dist} + {l}_{C} cos β )$$

$$where$$

$${α}_{min dist} = [ \begin{matrix}{tan}^{- 1} ( \frac{{l}_{C} sin β}{{x}_{DSV} - {l}_{C} cos β} ) & \begin{matrix}for \vert {l}_{T} sin ( α {\vert}_{\frac{\partial Dist}{\partial α} = 0} ) \vert + \\ \frac{{D}_{Knee}}{2} < \frac{U}{2}\end{matrix} \\ {sin}^{- 1} ( \frac{U - {D}_{Knee}}{2 {l}_{C}} ) & \begin{matrix}for \vert {l}_{T} sin ( α {\vert}_{\frac{\partial Dist}{\partial α} = 0} ) \vert + \\ \frac{{D}_{Knee}}{2} > \frac{U}{2}\end{matrix}\end{matrix} ] , \\ U = 2 \sqrt{{( \frac{D}{2} )}^{2} - {( h - \sqrt{{( \frac{D}{2} )}^{2} + {( \frac{B}{2} )}^{2}} )}^{2}}$$

$$and$$

$${h}_{2} = h - \sqrt{{( \frac{D}{2} )}^{2} - {( \frac{B}{2} )}^{2}}$$

In order to provide a useful image of the subject, in this case the Achilles tendon, it is important that the image slice direction is correctly aligned with the subject. In this case, it is preferable for the image slice to be perpendicular to the axis of the tendon. As the subject is moved, the motion controller 414 and the scanner host PC 426 are arranged to control the image slice orientation so that, as the leg is moved, and when it reaches its final position, the image slice direction is always in the required orientation relative to the imaged tendon.

In the embodiments described above the components were made of materials selected to have minimum effect on the MRI scanner in which they are located. The housing and gear wheels are all constructed of epoxy resin using rapid prototype stereo lithography processes. The ball bearings and rotor shaft are made of glass and the gearbox shafts are made of aluminium. Other materials may be selected, but they are preferably non-conductive and non-magnetic.

While the embodiments above comprise actuators for use in MRI compatible robots, it will be appreciated that actuators and robots according to the invention can be used in other (non-MR) applications areas where EM motors are undesirable or represent a safety risk. For example, in environments where explosion or fire hazards such as powder suspensions or flammable vapours are present.

The actuator can work at a wide range of pressures, depending on the design of the rotor. For medical applications, with systems in contact with the patient, the preferred values of pressure are around 1-1.5 bar, which is low enough to assure safety in case of leakage, malfunction etc. The turbine and gearbox can be designed to produce the required output speed and torque. For medical applications higher pressures may be acceptable, for example up to 2.0 bar. However, low pressures are generally desirable and pressures down to 0.5 bar can be used. For non-medical applications higher pressures still may be acceptable, for example up to 4.0 bar or even 5.0 bar or higher. Similarly rotor speeds will vary depending on the application, but the maximum turbine speed will generally be over 5,000 rpm, and indeed preferably over 10,000 rpm. Speeds in the range from 10,000 to 40,000 rpm will generally be suitable, and speeds from 15,000 to 30,000 will be preferable in some applications. Because of the very high gear ratios, the output shaft speed will generally be low, with a maximum of 1000 rpm, and for most applications a much lower maximum of 50, or even 10 rpm. Gear ratios will, as described above, generally need to be high. In general ratios of 1000:1 are preferable, with ratios of 2000:1 being most preferable in many applications.

The simplest way of limiting the air pressure to the values indicated above is for the air supply to be arranged to supply air at that pressure. This means that, even if the valves open fully, the maximum desired pressure will not be exceeded. However, in some cases it will be sufficient for the control system as a whole to control the air pressure supplied to the air inlet so as to limit it to the pressures indicated above.

1. An actuator comprising an input shaft, a turbine system coupled to the input shaft, gas inlet means arranged to direct gas flowing through it towards the turbine system to rotate the input shaft, an output shaft, and a gearing system connecting the input shaft to the output shaft so that the turbine system can drive the output shaft via the gearing system, wherein the gas inlet means defines two different flow paths for gas whereby the output shaft can be driven in both directions.

2. An actuator according to claim 1 wherein the turbine system comprises two sets of turbine blades and the gas inlet means is arranged to direct gas towards one of the sets to drive the input shaft in one direction, and against the other of the sets to drive the input shaft in the other direction.

3. An actuator according to claim 1 wherein the turbine system comprises a set of blades and the gas inlet means is arranged to direct gas towards one side of the blades to drive the input shaft in one direction, and towards the other side of the blades to drive the input shaft in the other direction.

4. An actuator according to claim 3 wherein the gas inlet means comprises first and second gas inlets and a first and second nozzles connected to the first and second inlets respectively and arranged to direct air from the first and second inlets onto first and second sides of the blades respectively.

5. An actuator according to claim 4 wherein each of the first and second gas inlets is connected to a plurality of nozzles.

6. An actuator according to claim 4 wherein the nozzles are tapered.

7. An actuator according to claim 2 wherein the turbine system comprises a turbine chamber in which the, or one of the, sets of blades is located, the actuator further comprising an outlet from the chamber which opens into the chamber at a point axially offset from the blades.

8. An actuation system comprising an actuator according to claim 1, and a control system arranged to control the supply of gas to the gas inlet means thereby to control the actuator.

9. An actuation system according to claim 8 wherein the control system comprises a gas supply, valve means arranged to control the flow of gas from the supply to the gas inlet means, and control means arranged to control the valve means thereby to control the actuator.

10. An actuation system according to claim 9 wherein the control means is arranged to receive a demand signal indicative of a demanded position of a control element driven by the actuator, and a position signal indicative of the actual position of the control element, and is further arranged to control the actuator to drive the control element towards the demanded position.

11. An actuation system according to claim 10 wherein the control means is arranged to detect a deceleration condition when deceleration of the control element is required, and to control the air inlet means to direct air towards the turbine system so as to produce a braking torque.

12. An actuator or actuation system according to claim 1 wherein the gearbox produces a gearing ratio of at least 1000:1 between the speeds of the input shaft and the output shaft.

13. An actuation system according to claim 8 wherein the gas supply is arranged to supply gas at a pressure of 5 bar or less.

14. An actuation system according to claim 13 wherein the gas supply is arranged to supply gas at a pressure of 2 bar or less.

15. An actuation system according to claim 8 wherein the control system is arranged to supply gas to the gas inlet at a pressure of 5 bar or less.

16. An actuation system according to claim 15 wherein the control system is arranged to supply gas to the gas inlet at a pressure of 2 bar or less.

17. An actuation system according to claim 8 wherein the control system is arranged to control the turbine system up to speeds of at least 10,000 rpm.

18. A robot system comprising a control element, an actuation system according to claim 8 arranged to control the position of the control element, and an operator input arranged to be controlled by a user to control the demand signal, whereby the user can control the position of the control element.

19. A robot system according to claim 18 wherein the control element is arranged to support a surgical device.

20. An MRI scanning system including a scanner volume and magnets arranged to vary the magnetic field in the scanner volume, and a robot system according to claim 18 wherein the actuator and the control element are located within the scanner volume.

21. A manipulator for a limb comprising a limb support arranged to support a limb of a subject, and a carrier system arranged to allow movement of the limb support in two orthogonal directions within a plane and rotation of the limb support about an axis.

22. A manipulator according to claim 21 further comprising three actuators arranged to provide movement of the limb support in the two directions and about the axis respectively.

23. An MRI scanning system comprising a manipulator according to claim 21, a scanner volume and magnets arranged to vary the magnetic field in the scanner volume, and control means arranged to control operation of the manipulator wherein the control means is arranged to define a set of conditions regarding movement of the limb within the scanner volume and to control operation of the manipulator so that those conditions are met.

24. A system according to claim 23 wherein the conditions at least partially define an end position of at least a part of the limb.

25. A system according to claim 23 wherein the conditions define an end orientation of at least a part of the limb.

26. A system according to claim 23 wherein the conditions define at least one extremity of the limb and a predetermined volume, and are arranged to control a trajectory of the extremity to ensure that it remains within the predetermined volume.

27. A system according to claim 23 wherein the limb comprises two parts and wherein the conditions define a set of acceptable positions or movements of the two parts relative to each other.

28. A system according to claim 23 wherein the control means is further arranged to define an image slicing direction of the scanner, and to control the image slicing direction so as to depend on the position of the limb.
